(19) United States
(12) Patent Application Publication
(10) Pub. No.: US 2022/0304347 A1
(43) Pub. Date: Sep. 29, 2022

(54) USE OF PROPOLIS OBTAINED BY SPECIAL EXTRACTION METHOD IN SPREADABLE MIXTURES

(71) Applicant: SBS BILIMSEL BIO ÇÖZÜMLER SANAYI VE TICARET A.S., Istanbul (TR)

(72) Inventors: Asli Elif TANUGUR SAMANCI, Istanbul (TR); Taylan SAMANCI, Istanbul (TR)

(21) Appl. No.: 17/433,696

(22) PCT Filed: Dec. 23, 2020
(86) PCT No.: PCT/TR2020/051362

§ 371 Date: Aug. 25, 2021

(30) Foreign Application Priority Data
Jun. 8, 2020 (TR) 2020/08794

(51) Int. Cl. A23L 21/20; A61K 8/98; A61K 8/9789; A61Q 19/00; B01D 11/02; B01D 19/00; B01D 1/22; B01D 9/00; A23D 7/005; B01D 21/26

(52) U.S. Cl. CPC A23L 21/20; A61K 8/988; A61K 8/9789; A61Q 19/00; B01D 11/0288; B01D 19/0078; B01D 1/222; B01D 9/0013; A23D 7/0056; B01D 21/262; A61K 2800/10; A23V 2002/00

(57) ABSTRACT

Disclosed is a spreadable propolis products which can be used in many sectors such as food, health and cosmetics that offer them for human consumption in their most suitable and in the highest form of bioavailability, in a fast and easy consumable state by properly extracting propolis which cause development of antioxidant, antimicrobial, antifungal, antivirus, anti-inflammatory, anti-cancerogen and anesthesic effects as well as many beneficial biological activities. Production methods thereof are also disclosed.

### TECHNICAL FIELD

The invention relates to propolis products.

The Invention relates in particular to spreadable propolis products which can be used in many sectors such as food, health and cosmetics that offer them for human consumption in their most suitable and in the highest form of bioavailability, in a fast and easy consumable state by property extracting propolis which cause development of antioxidant, antimicrobial, antifungal, antivirus, anti-Inflammatory, anti-cancerogen and anesthesic effects as well as many beneficial biological activities, and production methods thereof.

### PRESENT STATE OF THE ART

Raw propolis is an entirely natural bee product composed of phenolic and flavonoid substances which the bees collect from the leaves, stems and buds of plants. Raw propolis etymologically formed from the words Pro (Preliminary)+Polis (Protective) is the preliminary cleaner of the hive which is the habitat of bees. Bees cover the comb cells with raw propolis and then place their honey, larva, and food over it. Thus, the products produced by the bees are stored in the hive without deterioration. The bees produce propolis for protecting the hive against micro-organisms and external factors by repairing the cracks in the hive with propolis. It is known that propolis has antimicrobial, anti-inflammatory, antioxidant, anti-cancerogen and anesthesic effects and reinforces the immunological system. Therefore, it is being used frequently in medical, apitherapy, and healthy nutrition and biocosmetic fields.

Propolis which is produced by bees to protect their hives cannot be consumed as it is taken from the hive because it comprises high content of beeswax and parasites which try to reach the honey in the hive. In order for propolis to be converted to a suitable form for human consumption, it is necessary to remove the beeswax and impurities in its content. Bioavailability of propolis, which has 2% bioavailability in its raw state, can reach up 100% after removal of beeswax and other impurities. Our body employs propolis converted completely to pure form composed of antioxidant substances such as phenolic and flavonold.

Turkish patent application number TR 2020/05364 relates to propolis extract developed to be used in food, cosmetic and health fields, wherein lactic acid syrup used as the solvent comprises water to dilute propolis and lactic acid syrup. Said lactic acid syrup comprises lactic acid ranging in amounts from 20% to 85% by weight, and propolis extract comprises lactic acid syrup in an amount by weight from 30% to 99%, propolis in an amount of from 1% to 60% and water in an amount of from 5% to 30% by weight.

Turkish patent application number TR 2019/23111 which is another application present in the literature relates to production in tablet, granule, drop, lozenge, dermal cream, lipstick, shampoo, oral spray, dropper and/or chewing gum forms to be used in medicine, cosmetics and food sectors by processing raw propolis by obtaining both in liquid formulation and also in powder formulation by microencapsulation and so as to be suitable for industrial use. Liquid propolis extract and microencapsulated powder propolis obtained in the production stage have been extracted with the aid of ultrasonic homogenizer, ultrasonic bath or high pressure reactor. In order to microencapsulate the propolis, various gums, and cellulose compounds; and as formulation aids and nutrient input, inulin, casein, whey protein isolates and similar components have been used.

Although many products comprising propolis are being sold in the market, propolis content information of most of them is wrong, or extraction methods are insufficient. It is necessary for the raw propolis to be extracted correctly due to the indigestible components such as impurities and beeswax in its content, and to be supplied to consumption in its highest state of bioavailability by preserving the beneficial components in its content in the maximum amount. Antioxidant content and bioavailability of the products in the market are rather low.

Moreover, direct consumption of propolis is rather difficult due to its bitter taste and resinous structure. Therefore, products available in the market use many additives and chemicals to overcome this consumption difficulty. The purpose of using these chemicals is to bring in solubility to the resinous structure and to mask the bitter taste with the artificial sweeteners used. Moreover, the firms are unable to purify propolis correctly or import it from countries such as China. Residue and additive problems are frequently encountered in the imported propolis. Consequently, the firms are obliged to add chemical preservatives and additives to the final product due to the fact that they cannot make use of the antioxidant, antimicrobial, antifungal and similar bioactive effects of propolis. And this in turn causes the product to attain an undesirable condition.

Consequently, due to the abovementioned negativities and because of the insufficiency of the existing solutions about the subject it has been necessary to perform an improvement in the related technical field.

### BRIEF DESCRIPTION OF THE INVENTION

The present invention relates to spreadable propolis products which meet the abovementioned requirements, which eliminate all disadvantages and which offer some additional advantages, and its method of production and spreadable propolis product.

The primary purpose of the invention is to provide the method to obtain a properly extracted liquid propolis extract with high bioavailability and spreadable propolis product.

A purpose of invention is to provide liquid propolis extract having no side effect or drug food interaction, and to provide spreadable propolis product.

Another purpose of invention is to form the steps applied during the method in a way not to disturb the stability of propolis product in a suitable and efficient result manner.

Another purpose of invention is to obtain properly extracted spreadable propolis extract with high flavonoid content which cause development of antioxidant, antimicrobial, antifungal, antivirus, anti-inflammatory, anti-cancerogen and anesthesic effects as well as many beneficial biological activities.

Another purpose of invention is to provide raw materials or final products from the obtained spreadable propolis extract to be used for healthy nutrition, apitherapy and biocosmetics purposes in the food, health and cosmetics sectors.

In order to accomplish the above mentioned purpose, the invention relates to a method of obtaining spreadable propolis product which comprises the following process steps and which has high antioxidant capacity (a) disintegrating the raw propolis by passing it through a grinder; (b) mixing the disintegrated propolis with a solvent selected from a group consisting of ethyl alcohol, propylene glycol, ultra pure water, carob extract and fruit juice; (c) mixing the obtained solution by adding lemon juice or apple cider vinegar as acid regulator; (d) shaking the degassed solution; (e) degassing the shaken solution in ultrasonic bath; (f) centrifuging the degassed solution; (g) removal of volatile substances by drying the centrifuged mixture in a rotary evaporator until the extent of dryness; (h) carrying out analysis; and comprises the process steps of
(i) Addition of at least one fruit juice, honey, spices and/or royal jelly to the mixture in which solvents have been removed by drying and mixing the same;
(j) Addition of perga, pollen, spices and/or snacks (preferably hazelnuts, peanuts, pistachios, cocoa, carob) to the mixture;
(k) Mixing the mixture in a homogenizer;
(l) Degassing it in an ultrasonic bath;
(m) Formation of crystals by keeping the degassed mixture in a freezer preferably for 1 day.

The invention also includes the propolis products obtained by the above mentioned method and the use of these products for healthy nutrition, apitherapy and biocosmetic purposes in food, health and cosmetics sectors.

Structural and characteristic features of the invention and all of its advantages will be understood more clearly by means of the detailed description given below and therefore it should be appreciated by considering this detailed description.

### DETAILED DESCRIPTION OF THE INVENTION

In this detailed description, spreadable propolis products of the invention and production methods thereof are described aimed at only better understanding of the subject and without forming any limiting effects.

The Invention relates in particular to spreadable propolis products which can be used in many sectors such as food, health and cosmetics that offer them for human consumption in their most suitable and in the highest form of bioavailability, in a fast and easy consumable state by property extracting propolis which cause development of antioxidant, antimicrobial, antifungal, antivirus, anti-inflammatory, anti-cancerogen and anesthesic effects as well as many beneficial biological activities, and production methods thereof.

The invention also relates to a spreadable propolis product suitable for healthy nutrition, apitherapy and biocosmetic purposes for use in food, health and cosmetics sectors offered as drops, syrups or drinks wherein the spreadable propolis extract obtained by the extraction of pure propolis with a solvent selected from the group consisting of ethyl alcohol, propylene glycol, ultra pure water, carob extract and fruit juice and lemon juice or apple cider vinegar as acid regulator comprises honey, royal jelly, perga, pollen, snacks and/or spices.

Since the raw propolis cannot be digested directly due to the indigestible components such as impurities and beeswax in its content; it has to be freed of the impurities by extracting with a solvent. In Table 1, preferred raw materials of the invention in the method of invention and their amounts are given. The spreadable propolis product which has been developed accordingly comprises:
honey preferably in an amount from 12.5% to 37.5% (more preferably 25%) based on total weight,
snacks preferably in an amount from 2.5% to 7.5% (more preferably 5%) based on total weight,
royal jelly preferably in an amount from 0.5% to 2.5% (more preferably 1%) based on total weight,
spices preferably in an amount from 0.05% to 0.5% (more preferably 0.25%) based on total weight,
pollen preferably in an amount from 0.5% to 2.5% (more preferably 1%) based on total weight and/or
perga preferably in an amount from 0.5% to 2.5% (more preferably 1%) based on total weight.

TABLE 1
Raw materials used in the method of the invention

| | | Usable | Preferred | Usable |
|---|---|---|---|---|
| | Preferred | amounts | amount | amount |
| | amounts | (g) for | (%) | (%) |
| | (g) | each | for each | for each |
| | for each | 10-50 | 10-50 | 10-50 |
| | 20 grams | grams of | grams | grams of |
| | of raw | raw | of raw | raw |
| Usable | propolis | propolis | propolis | propolis |
| solvents | extraction | extraction | extraction | extraction |
| Ethyl | 500 | 250-750 | 25 | 12.5-37.5 |
| alcohol | | | | |
| Propolis | 20 | 10-50 | 1 | 0.5-2.5 |
| Propylene | 500 | 250-750 | 25 | 12.5-37.5 |
| glycol | | | | |
| Ultra pure | 500 | 250-750 | 25 | 12.5-37.5 |
| water | | | | |
| Fruit | 500 | 250-750 | 25 | 12.5-37.5 |
| Concentrate | | | | |
| Carob | 500 | 250-750 | 25 | 12.5-37.5 |
| extract | | | | |
| Usable acid | | | | |
| regulators | | | | |
| Lemon juice | 20 | 10-50 | 1 | 0.5-2.5 |
| Apple cider | 20 | 10-50 | 1 | 0.5-2.5 |
| vinegar | | | | |
| Other Raw | | | | |
| Materials | | | | |
| Honey | 500 | 250-750 | 25 | 12.5-37.5 |
| Snacks | 100 | 50-150 | 5 | 2.5-7.5 |
| Royal Jelly | 20 | 10-50 | 1 | 0.5-2.5 |
| Spices | 5 | 1-10 | 0.25 | 0.05-0.5 |
| Pollen | 20 | 10-50 | 1 | 0.5-2.5 |
| Perga | 20 | 10-50 | 1 | 0.5-2.5 |

Raw propolls: Propolis is a plantal substance in the form of a sticky resin which is collected by bees from the trees and carried to the hives. It is a bee product having natural antibiotic property. Propolls which has a natural antibiotic property causes implementation of antioxidant, antimicrobial, antifungal, antivirus, anti-inflammatory, anti-cancerogen and anesthesic effects as well as many useful biological activities. It strengthen the immune system. It is known to be effective against cancer, inflammatory diseases, diseases of viral and bacterial origin. Raw propolis should be freed of the impurities by extraction since it cannot be directly consumed due to the indigestible components such as impurities and beeswax in its content. Therefore, raw propolis should be extracted with the correct solvents.

Solvent: Raw propolis should be freed of the impurities by extraction since it cannot be directly consumed due to the indigestible components such as impurities and beeswax in its content. Solvent is the agent which provides the dissolution of the raw propolis and transport of the active materials. In the method of obtaining the propolis extract of the invention, food grade ethanol with a purity of over 50% (ethanol), propylene glycol, at least one fruit juice, carob extract or ultra pure water are being used as the solvent. Different solvents which are not harmful against human health according to the form and composition of the desired final product to be obtained by fruit juice, pomegranate juice, orange juice, strawberry juice, apple juice, peach juice, sour cherry juice or similar fruit juices mentioned herein can be used. Solvents which are not suitable for human health such as methanol, ethyl acetate, chloroform and hexane are not preferred in the invention.

Ethanol, ethyl alcohol or plantal alcohol is a colorless chemical compound obtained from plants. It is currently being used as solvent in vitamin syrups.

Ultra pure water is the more purified state of pure water. Pure water is the water in which mineral substances present in the water have been removed. And ultra pure water is water with a conductivity value of 0.055 μS/cm (25° C.) or electrical resistivity of 18.2 Megaohm-cm.

Propylene glycol is a compound of a derivative of mineral oil having many fields of usage in the commercial industry. It is odorless and it does not have any certain taste definition. Propylene glycol is being used in food, cosmetics, pharmaceutical and plastics industries. Ethyl alcohol and propylene glycol are suitable to be used in foods and are not subjected to any dissolver heating process.

Orange juice can reduce blood pressure as well as “bad” cholesterol levels and as a consequence of which can prevent cardiovascular problems. It is rather rich source for vitamin C which is ideal for preventing common cold. At the same time, it aids in regulating the intestinal system.

Pomegranate juice prevents cholesterol and regulates blood sugar and prevents its increase. It regulates blood pressure. It protects cardiac health, and supports regular functioning of it. It protects the body resistance against infections and increases it. It provides removal of toxins by its diuretic action. It helps smooth appearance of the skin.

Strawberry juice has diuretic, purifying and antioxidant properties. It is rich in vitamin A, C and E, and iron, vitamin K and folic acid. There are soluble fibers inside strawberries, that is, it is an excellent aid for the absorption of carbohydrates and it stabilizes blood sugar level. It protects the heart and is effective against constipation.

Sour cherry juice has powerful antioxidant, antidiabetic, anti-inflammatory and obesity preventing effects depending on the presence of powerful anthocyanins. There is plenty of Vitamin A, Vitamin E, B group of vitamins and Vitamin K in sour cherry. There is also phosphorus, magnesium, copper, calcium, potassium and sodium in sour cherry.

Apple juice is an important source of vitamin C. Vitamin C which is a very powerful antioxidant renews the cells and strengthens the immunological system. It protects the body against infections especially during winter months. It is a vitamin that cannot be produced by the body and has to be taken via foods. It also comprises vitamins K, A, E and 86. It is known to be effective against diabetes. It has a therapeutic effect to chronic constipation disorder thanks to its high fiber content.

Peach juice is particularly rich in vitamins A, C, E and K; it has benefits such as relieving from obesity, cholesterol, neurodegenerative disease as well as has important contribution to eye care, skin care, a healthy nervous system, protection of bones and teeth. Peaches are also known to possess anti-cancerous properties.

Acid regulating substance is being used to increase the acidity of the solution consisting of propolis and solvent in the method of obtaining high flavonoid content propolis extract of the invention. When ethanol, propylene glycol or ultra pure water mixture is being used as solvent, preferably 1 ml of lemon juice can be used as acid regulating substance. When propylene glycol, carob extract or at least one of the above mentioned fruit juices is being used as solvent, preferably 1 ml of apple cider vinegar can be used as acid regulating substance. Different acid regulators too can be preferred for different solvents other than these.

Ginger, turmeric and cinnamon are preferably being used as spices. There is a great number of published reports related to the antioxidative activity of these spices. Spices are food supplements that have been used as flavoring and coloring agents and as preservatives for thousands of years. The digestion stimulating effect, hypolipidemic effect, antidiabetic effect, antilithogenic property, antioxidant potential, anti-inflammatory property, antimutagenic and anticarcinogenic properties of the spices have been documented. Among these, hypocholesterolemic and antioxidant properties of a few specific spices have a wide range nutraceutical value. These useful physiological effects also have possible therapeutical treatment potential under various disease conditions. Turmeric (Curcuma longa) is used in many urinary diseases, liver diseases and in jaundice. Turmeric extract and curcumin isolated from it are rather cytotoxic in mammalian cells and are effective in reducing animal tumors and shows potential of usage in cancer treatment. Ginger (Zingiber officinale Rosc.) is one of the richest sources of antioxidants which cleanse the free radicals and the health inconsistencies that they bring along with them. Ginger comprises gingerois as active components which exhibit antioxidant activity determined by in vitro studies.

Snacks are an important group food having health protective effects because of comprising dried nuts with hard shells, mono unsaturated fatty acids, non-lipid components, plantal protein; being a source of soluble roughage; being rich in minerals and vitamins such as magnesium, potassium, zinc and selenium. Dried nuts have been shown to possess substances which considerably reduce coronary heart disease, some cancer kinds and some other disease and physiological conditions and syndrome risks. Compounds determined with positive health benefits comprise vitamin E, vitamin B6, mono unsaturated fatty acids (essentially oleic acid) and sterols for their antioxidant properties.

Pollen is a product of a great variety and rich in biologically active substances. In pollen grains from different plant kinds 200 substances have been found. Proteins, amino acids, carbohydrates, lipids and fatty acids, phenolic compounds, enzymes and coenzymes, vitamins and bio-elements are included in the fundamental chemical substances.

Bee pollen is used in apitherapeutical treatment since it shows a series of actions such as antifungal, antimicrobial, antiviral, antiinflammatory, immunoregulatory and local analgesic action and also because it facilitates the granulation process of burn wound healing. Bee pollen is a precious nutritive substance and a natural product of honey bee advertised as a source of energy. The health promoting value of bee pollen originates from the presence of enzymes and co-enzymes in the bee pollen as well as secondary plant metabolites (tocopherol, niacine, thyamine, biotine and folic acid, polyphenols, carotenoid pigments, phytosterols).

Royal jelly is a very precious bee product having a great number of functional properties such as antibacterial activity, anti-inflammatory activity, vasodilatative and hypotensive activities, disinfectant effect, antioxidant activity, antihypercholesterolemic activity and antitumor activity.

Perga is pollen in the form of pellets collected by the bees and carried back to the hive by mixing them with their own digestive enzymes, comprising a little amount of honey and beeswax. By means of fermentation implemented by the enzymes of the bees, perga which is formed by melting of the outer membrane of the pollens encompasses many useful microorganisms thereby showing probiotic property. Perga which comprises many vitamins, phenolics and antioxidants is also being used as a natural food supplement.

The invention comprises honey as a sweetener. Honey is a natural product which is modified by combining with intrinsic substances after plant nectars, secretions in the live parts of plants or secretions of insects that live on the live parts of plants are collected by the honey bee, which is reduced in its water content and which is ripened by storing in the honeycomb. Honey which is a source of vitamins and minerals has a lot of benefits to the body especially in the immunological system.

The method of obtaining spreadable propolis product of the invention comprising the process steps of (a) disintegrating the raw propolis in amounts indicated in Table 1 by passing it through a grinder; (b) mixing the disintegrated propolis with a solvent selected from a group consisting of ethyl alcohol, propylene glycol, ultra pure water, carob extract and fruit juice, preferably in amounts indicated in Table 1; (c) mixing the obtained solution by adding lemon juice or apple cider vinegar as acid regulator; (d) shaking the degassed solution; (e) degassing the shaken solution in ultrasonic bath; (f) centrifuging the degassed solution; (g) removal of volatile substances by drying the centrifuged mixture in a rotary evaporator until the extent of dryness; (h) carrying out analysis and having a high antioxidant capacity in its most fundamental form comprises the following process steps;
(n) Adding at least one fruit juice, honey, spices and/or royal jelly to the mixture in which solvents have been removed by drying, and mixing the same;
(o) Addition of perga, pollen spices and/or snacks (preferably hazelnuts, peanuts, pistachios, cocoa, carob) to the mixture;
(p) Mixing the mixture in a homogenizer;
(q) Degassing in an ultrasonic bath;
(r) Formation of crystals by keeping the degassed mixture in a freezer preferably for 1 day.

In an embodiment of the invention, when the above mentioned ethanol, propylene glycol or ultra pure water is used as solvent, lemon juice can be used as acid regulating substance in the amounts given in Table 1. When propylene glycol, carob extract or at least one of the above mentioned fruit juices is used as solvent, apple cider vinegar can be used as acid regulating substance in the amounts given in Table 1.

A sample is taken from the supernatant which is formed following the centrifuging process, and bioavailable phenolic flavonoid substance content which the propolis comprises is analyzed in LC-MSMS (Liquid Chromatography-Mass/Mass Spectrometer). In the analysis results, the condition of galangine, pinocembrin, crisine, cinnamic acid to be above 10 mg/g and p—coumaric acid, ferulic acid, cafeic acid phenethyl ester (CAPE) to be above 5 mg/g is sought. In the event when the analysis results are above the desired values, dry matter determination of the sample is carried out. In the event when the analysis results are not suitable, said solution is again subjected to the processes of continuous stirring in a medium temperature of 20-30° C. for at least 7 days in darkness; degassing in an ultrasonic bath for 3 hours at 25° C.; centrifuging at 10 000 g for 20-30 minutes, in sequence. A new sample is taken from the supernatant which forms as a result of centrifuging process and phenolic flavonoid substance content is analyzed again in LC-MSMS.

In the obtained solution mentioned above, if the amount of dry substance has reached the desired ratio, fruit juice, honey, spices and/or royal jelly are added to the mixture in which solvents have been removed by drying and the mixture is stirred. Perga, pollen spices and/or snacks (preferably hazelnuts, peanuts, pistachios, cocoa, and carob) are added to the mixture. The mixture is stirred in a homogenizer preferably at 30° C. preferably for 15-30 minutes and is degassed in an ultrasonic bath. Crystals are formed finally upon keeping the degassed mixture in the freezer preferably for 1 day. Following the completion of the process, filling and packaging processes are carried out. Useful components that are present in the propolis are preserved by the described process, and products which have high bioavailability, which are suitable for human consumption, in spreadable form and in consumption ways can be produced.

1. A Method of obtaining spreadable propolis product comprising the process steps of (a) disintegrating raw propolis by passing it through a grinder; (b) mixing the disintegrated propolis with a solvent selected from a group consisting of ethyl alcohol, propylene glycol, ultra pure water, carob extract and fruit juice; (c) mixing the obtained solution by adding lemon juice or apple cider vinegar as an acid regulator; (d) shaking the degassed solution; (e) degassing the shaken solution in an ultrasonic bath; (f) centrifuging the degassed solution; (g) removal of volatile substances by drying the centrifuged mixture in a rotary evaporator until the extent of dryness; (h) carrying out analysis, and having high antioxidant capacity, further comprising the process steps of:
(j) addition of at least one fruit juice, honey, spices and/or royal jelly to the mixture in which solvents have been removed by drying and mixing the same;
(k) addition of perga, pollen, spices and/or snacks to the mixture;
(l) mixing the mixture in a homogenizer;
(m) degassing it in an ultrasonic bath;
(n) formation of crystals by keeping the degassed mixture in the freezer.

2. The method according to claim 1, wherein in process step (b), each 10-50 g of disintegrated propolis is mixed with 250-750 grams of solvent selected from a group consisting of ethyl alcohol, propylene glycol, ultra pure water, carob extract and fruit juice.

3. The method according to claim 1, wherein the fruit juice is selected from a group consisting of pomegranate juice, orange juice, strawberry juice, apple juice, peach juice and sour cherry juice.

4. The method according to claim 1, wherein in process step (c), 10-50 grams of lemon juice or apple cider vinegar is added to each 10-50 grams of solution comprising disintegrated propolis and stirred.

5. The method according to claim 1, wherein in process step (e), the solution is shaken in a dark medium at a temperature of 20-30° C. for 7 days.

6. The method according to claim 1, wherein in process step (f), the solution is degassed for 3 hours at 25° C. in an ultrasonic bath.

7. The method according to claim 1, wherein in process step (g), the solution is centrifuged at 10 000 G for 20 minutes.

8. The method according to claim 1, wherein in process step (i), phenolic flavonoid substance content of the sample taken from the supernatant which forms as a result of centrifuging is analyzed in LC-MSMS, and if phenolic flavonoid substance amount in the analysis results is at least in the value of 10 mg/g of galangine, pinocembrine, crisine, cinnamic acid and at least 5 mg/g of p—coumaric acid, ferulic acid, cafeic acid fenetil ester, dry substance determination in the extract obtained after centrifuging is carried out; if analysis results are not at desired values, process steps ‘d, e, f’ are repeated in sequence.

9. The method according to claim 1, wherein said spices are selected from a group consisting of ginger, thyme, cinnamon and turmeric.

10. The method according to claim 1, wherein in process step (1), the mixture is stirred at 30° C. for 15-30 minutes in a homogenizer.

11. The method according to claim 1, wherein in process step (j), 250-750 grams of honey and/or 10-50 grams of royal jelly are added.

12. The method according to claim 1, wherein in process step (k), 10-50 grams of perga, 10-50 grams of pollen, 50-150 grams of snacks and/or 1-10 grams of spices are added.

13. The method according to claim 1, wherein said snacks are selected from a group consisting of hazelnuts, peanuts, pistachios, cocoa and carob.

14. The method according to claim 1, wherein in process step (m), the mixture is kept in a freezer for 1 day.

15. Spreadable propolis products obtained by the method according to claim 1.

16. The use of products according to claim 15 for healthy nutrition, apitherapy, biocosmetic purposes in food, health and cosmetics sectors.

17. A spreadable propolis product offered as drops, syrups or drinks for healthy nutrition, apitherapy, biocosmetic purposes in food, health and cosmetics sectors, wherein the pure propolis comprises a solvent selected from the group consisting of ethyl alcohol, propylene glycol, ultra pure water, carob extract and fruit juice, and wherein it comprises liquid propolis extract obtained by extracting it with lemon juice or apple cider vinegar, honey, royal jelly, perga, pollen, snacks and/or spices as acid regulator.

18. The spreadable propolis product according to claim 17, comprising honey in an amount from 12.5% to 37.5% based on total weight.

19. Liquid propolis product according to claim 18 wherein a honey ratio is 25%.

20. Liquid propolis product according to claim 17, comprising royal jelly in an amount from 0.5% to 2.5% based on total weight.

21. Liquid propolis product according to claim 20 wherein a royal jelly ratio is 1%.

22. Liquid propolis product according to claim 17, comprising spices in an amount from 0.05% to 0.5% based on total weight.

23. Liquid propolis product according to claim 22, wherein a spices ratio is 0.25%.

24. Liquid propolis product according to claim 17, comprising pollen in an amount of from 0.5% to 2.5% based on total weight.

25. Liquid propolis product according to claim 24, wherein a pollen ratio is 1%.

26. Liquid propolis product according to claim 17, comprising perga in an amount from 0.5% to 2.5% based on total weight.

27. Liquid propolis product according to claim 26, wherein a perga ratio is 1%.

28. Liquid propolis product according to claim 17, comprising snacks in an amount of from 2.5% to 7.5% based on total weight.

29. Liquid propolis product according to claim 28, wherein a snacks ratio is 5%.
